(19) United States
(12) Patent Application Publication
(10) Pub. No.: US 2007/0059274 A1
(43) Pub. Date: Mar. 15, 2007

(54) Ophthalmic compositions containing a PVA/borate gelling system

(76) Inventors: Bahram Asgharian, Arlington, TX (US); David L. Meadows, Collyville, TX (US)

Correspondence Address: ALCON, IP LEGAL, TB4-8, 6201 SOUTH FREEWAY, FORT WORTH, TX, 76134, US

(21) Appl. No.: 11/601,165

(22) Filed: Nov. 17, 2006

Related U.S. Application Data
(63) Continuation of application No. 11/000,725, filed on Dec. 1, 2004.

(51) Int. Cl. A61K 31/74

(52) U.S. Cl. 424/78.18

(57) ABSTRACT

Ophthalmic compositions containing a polyvinyl alcohol/borate gelling system are described. The system forms a gel or partial gel when the composition is applied to the eye. The compositions are particularly useful as ocular lubricants or artificial tears.

### CROSS-REFERENCE TO RELATED APPLICATION

This application is a Continuation (CON) of co-pending U.S. application Ser. No. 11/000,725, filed Dec. 1, 2004, priority of which is claimed under 35 U.S.C. §120, the contents of which are incorporated herein by reference. This application also claims priority under 35 U.S.C. §119 to U.S. Provisional Patent Application No. 60/528,603, filed Dec. 11, 2003, the contents of which are incorporated herein by reference.

### BACKGROUND OF THE INVENTION

The present invention is directed to ophthalmic compositions that form a gel when applied to the eye. The transformation of the compositions from a solution to a gel is based on the presence of a polyvinyl alcohol/borate gelling system in the compositions. The compositions are particularly adapted for use as ocular lubricants or artificial tears in humans.

Various types of gelling systems for ophthalmic compositions have been described in the prior art:

U.S. Pat. No. 4,136,173 (Pramoda, et al.) discloses the use of therapeutic compositions containing xanthan gum and locust bean gum which are administered in liquid form and gel upon instillation. This reference describes a mechanism for transition from liquid to gel involving pH change. pH sensitive gels such as carbomers, xanthan, gellan, and those described above, need to be formulated at or below the pKa of their acidic groups (typically at a pH of about 2 to 5). Compositions formulated at low pH, however, are irritating to the eye.

The use of locust bean gum to form a gel vehicle for ophthalmic drug delivery is described in U.S. Pat. No. 4,136,177 (Lin, et al.). However, the gels described by Lin, et al. are formed at the time of manufacture, rather than upon application to the eye.

U.S. Pat. No. 4,861,760 (Mazuel, et al.) discloses ophthalmic compositions containing gellan gum which are administered to the eye as non-gelled liquids and gel upon instillation due to a change in ionic strength. These systems do not involve the use of small cross-linking molecules, but instead provide gel characteristics due to self cross-linking during ionic condition changes.

Gels involving the cross-linking of polysaccharides with borates are disclosed for use as well fracturing fluids in U.S. Pat. No. 5,082,579 (Dawson), U.S. Pat. No. 5,145,590 (Dawson), and U.S. Pat. No. 5,160,643 (Dawson). These patents describe the use of borates and polysaccharides for industrial oil well excavation.

The use of galactomannans (e.g., hp-guar) in ophthalmic compositions, including ocular lubricant and artificial tear compositions, is described in U.S. Pat. No. 6,583,124 (Asgharian). An ocular lubricant eye drop based on the invention described therein is sold under the name “SYSTANE™” by Alcon Laboratories, Inc.

Polyvinyl alcohol is a synthetic polymer. It has been widely used in pharmaceutical products, including ophthalmic compositions. For example, it has been used in solutions for treating hard contact lenses, as well as in artificial tear products, such as HypoTears PF (Novartis Ophthalmics) and ReFresh (Allergan). However, the existing commercial artificial tear products that contain polyvinyl alcohol (“VA”) do not contain a PVA/borate gelling system of the type described herein.

The use of a PVA/borate gelling system to form topical ophthalmic gels for delivery of various drugs to the eye is described in U.S. Pat. No. 4,255,415 (Chrai, et al.). However, the '415 patent does not disclose ophthalmic solutions containing PVA and borate that form gels upon application to the eye, nor does it describe the use of such gel-forming solutions as ocular lubricants or artificial tears.

WIPO Publication No. WO 94/10976 (Goldenberg et al.) discloses a low pH PVA-borate delivery system that does go through liquid/gel transition. This system has the disadvantage, however, of limited gelling effects, and only at certain concentrations of PVA depending on the molecular weight of the PVA utilized.

The use of borate/polyol complexes to formulate ophthalmic compositions containing PVA is described in U.S. Pat. No. 5,505,953 (Chowhan). The '953 patent does not disclose PVA/borate gelling systems of the type described and claimed herein. This patent teaches a method to overcome the incompatibility of PVA with borates by addition of a monomeric polyols such as mannitol or sorbitol.

### SUMMARY OF THE INVENTION

The present invention is directed to the ophthalmic compositions that contain a gelling system comprising a polyvinyl alcohol polymer or copolymer and a borate cross linker. The compositions are formulated and manufactured as liquids or partially gelled liquids that thicken to form gels upon application to the eye. The compositions of the present invention are particularly useful as artificial tears or ocular lubricants, but may also be utilized to deliver ophthalmic drugs to the eye. The gel-forming ability of the PVA/borate systems of the present invention provides better ocular lubricity and enhances the retention of the compositions in the eye, as compared to non-gelling PVA compositions.

### BRIEF DESCRIPTION OF THE DRAWING

FIG. 1 is a graph showing viscosity as a function of pH for the PVA/borate composition described in Example 2.

### DETAILED DESCRIPTION OF THE INVENTION

The compositions of the present invention will contain an amount of a PVA/borate gelling system sufficient to form a gel or partial gel upon application of the compositions to the eye.

The gelling system includes one or more polymers or copolymers containing polyvinyl alcohol. The preferred polymers and copolymers have a molecular weight of 10,000 Daltons or higher. The preferred polymers are vinyl alcohol-vinyl pyrrolidone block copolymers. Such polymers and copolymers containing PVA are commercially available. The commercially available PVA polymers and copolymers have varying degrees of hydrolysis of vinyl acetate. The preferred grades of polymers and copolymers are hydrolyzed to an extent of 98% or higher, and have molecular weights of greater than 50,000 Daltons.

The borate compounds which may be used in the compositions of the present invention are boric acid and pharmaceutically acceptable salts thereof, such as sodium borate (borax) and potassium borate. As used herein, the term “borate” refers to boric acid and all pharmaceutically suitable salts of boric acid. Borates are common excipients in ophthalmic formulations due to good buffering capacity at physiological pH and well known safety and compatibility with a wide range of drugs and preservatives. Borates also have inherent bacteriostatic and fungistatic properties, and therefore aid in the preservation of the compositions.

The compositions of the present invention will contain one or more PVA polymers or copolymers and one or more borates in an amount sufficient to form a gel or partial gel when the composition is applied to the eye. The amount of polymer or copolymer and borate required for a particular composition will be determined based on various factors, such as the molecular weight and/or grade of the particular polymer or copolymer selected and the type of gelling properties desired.

The borate or polymer concentration may be manipulated in order to arrive at the appropriate viscosity of the composition upon gel activation (i.e., after administration to the eye). If a strongly gelling composition is desired, then the borate or polymer concentration may be increased. If a weaker gelling composition is desired, such as a partially gelling composition, then the borate or polymer concentration may be reduced. Other factors may influence the gelling features of the compositions of the present invention, such as the nature and concentration of additional ingredients in the compositions, e.g., salts, preservatives, chelating agents and so on.

The preferred non-gelled compositions of the present invention, i.e., compositions not yet gel-activated by the eye, will generally have a viscosity of from about 5 to 1000 cps. The preferred gelled compositions of the present invention, i.e., compositions gel-activated by the eye, will generally have a viscosity of from about 50 to 50,000 cps.

The compositions of the present invention will typically contain one or more PVA polymers or copolymers in an amount of from about 0.1 to 5% weight/volume (“w/v”), and borate in an amount of from about 0.01 to 2% (w/v). Preferably, the compositions will contain 0.5 to 3.0% (w/v) of one or more PVA polymers or copolymers and 0.1 to 1.0% (w/v) of a borate compound. Most preferably, the compositions will contain 0.8 to 2.0% (w/v) of one or more PVA polymers or copolymers and 0.25 to 1.0% (w/v) of a borate compound.

The PVA/borate gelling characteristics described herein can be customized by using a second polymeric material, such as povidone or cellulose derivatives (e.g., HEC, HPMC and others). Alternatively, non-polymeric polyols such as mannitol or sorbitol can be incorporated to limit the gel forming ability of a composition. The compositions of the present invention can additionally contain one or more antimicrobial agents to preserve the composition from microbial contamination, as well as essential ions found in human tears. Conditioning or comfort drop compositions for contact lenses according to this invention may additionally contain one or more surfactants to remove deposits from contact lenses.

Combinations of the gelling system of the present invention and prior gelling systems is also contemplated by the present invention. Such prior gelling systems may include ionomers, such as xanthan, gellan, carageenan and carbomers, and thermogels, such as ethylhydroxyethyl cellulose or galactomannan/borate gelling systems of the type described in U.S. Pat. No. 6,583,124 (Asgharian).

Other ingredients may be added to the compositions of the present invention. Such ingredients generally include tonicity adjusting agents, chelating agents, active pharmaceutical agent(s), solubilizers, preservatives, pH adjusting agents and carriers. Other polymer or monomeric agents such as polyethylene glycol and glycerol may also be added for special processing. Tonicity agents useful in the compositions of the present invention may include salts such as sodium chloride, potassium chloride and calcium chloride; non-ionic tonicity agents may include propylene glycol and glycerol; chelating agents may include EDTA and its salts; solubilizing agents may include Cremophor EL® and tween 80; other carriers may include amberlite® IRP-69; pH adjusting agents may include hydrochloric acid, Tris, triethanolamine and sodium hydroxide; and suitable preservatives may include polyquaternium-1 and polyhexamethylene biguanide. The above listing of examples is given for illustrative purposes and is not intended to be exhaustive. Examples of other agents useful for the foregoing purposes are well known in ophthalmic formulation and are contemplated by the present invention.

The compositions of the present invention may be used to lubricate the eye or provide artificial tear solutions to treat, for example, dry eye. In general, artificial tear solutions will contain tonicity agents, polymers and preservatives, as described above.

The compositions of the present invention are primarily adapted for use as artificial tears or ocular lubricants. However, the compositions may also be utilized to administer various pharmaceutically active compounds to the eye. Such pharmaceuticals may include, but are not limited to, anti-hypertensive, anti-glaucoma, neuro-protective, anti-allergy, muco-secretagogue, angiostatic, anti-microbial, pain relieving, anti-inflammatory agents, and active pharmaceutical agents to treat dry eye (e.g., corticosteroids, such as rimexolone).

Examples of pharmaceutically active agents which may be included in the compositions of the present invention, and administered via the methods of the present invention include, but are not limited to: glaucoma agents, such as betaxolol, timolol, pilocarpine, brimonidine, carbonic anhydrase inhibitors and prostaglandins; dopaminergic antagonists; post-surgical antihypertensive agents, such as para-amino clonidine (apraclonidine); anti-infectives, such as moxifloxacin, levofloxacin, gatifloxacin, ciprofloxacin and tobramycin; non-steroidal and steroidal anti-inflammatories, such as naproxen, diclofenac, suprofen, ketorolac, tetrahydrocortisol and dexamethasone; proteins; growth factors, such as epidermal growth factor; and anti-allergics.

The following Examples are provided to further illustrate the present invention:

### EXAMPLE 1

The formulation is an example of an artificial tear composition according to this invention:

| Ingredient | W/V % |
|---|---|
| Polyvinyl Alcohol | 1.5 |
| Boric Acid | 0.5 |
| Sodium Chloride | 0.1 |
| Potassium Chloride | 0.12 |
| Calcium Chloride | 0.0053 |
| Magnesium Chloride | 0.0064 |
| Zinc Chloride | 0.00015 |
| Polyquaternium-1 | 0.0005 |
| Sodium Hydroxide | pH to 6.5-7.0 |
| Purified Water | Qs to 100 |

The composition is prepared in two parts. A first part (“Part I”) is prepared by dispersing PVA in 50% of the batch volume of hot purified water and allowing the polymer to hydrate. The resulting solution is then autoclaved at 121° C. for 35 minutes, and mixed while cooling. A second part (“Part II”) is prepared by dispersing the remaining ingredients in 40% of the batch volume of purified water, allowing the ingredients to dissolve, and then adjusting the pH to near the target pH. The Part II solution is sterile filtered through a 0.2 micron sterilizing filter and then added slowly to the Part I solution while stirring to minimize local gelation.

The above-described composition is a liquid in the bottle, which allows for ease of dispensing. Upon application of a small amount (e.g., 1 to 2 drops) of the composition to the eye, a soft fluid gel is formed upon slight increase in pH. The gel offers increased retention, relative to conventional ophthalmic solutions, and provides excellent lubrication to the eye.

### EXAMPLE 2

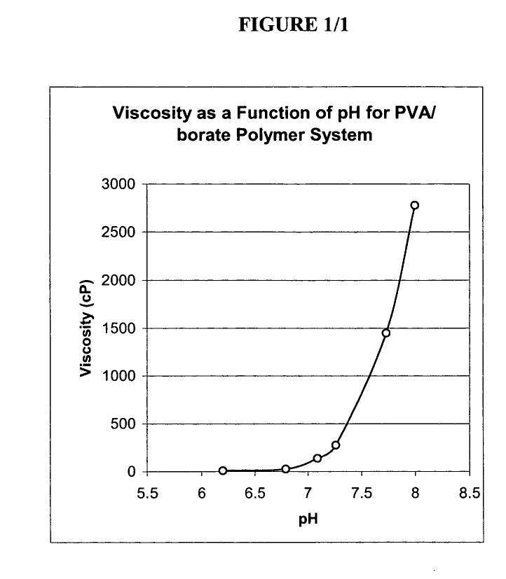

A solution of polyvinyl alcohol at 1.8% and boric acid at 0.5% was prepared by: dissolving the polymer in hot water and allowing it to disperse; adding boric acid from a stock solution, dropwise, while mixing; and adjusting the pH with 1N sodium hydroxide solution. The viscosity with increase in pH is plotted in FIG. 1: The solution exhibited strong gelling properties at a pH of from 6 to 8.

We claim:

1. An ophthalmic composition comprising a polyvinyl alcohol/borate gelling system, said gelling system containing a polyvinyl alcohol polymer or copolymer and a borate compound in amounts sufficient to form a gel or partial gel when the composition is topically applied to the eye.

2. A method of lubricating the eye of a human patient, which comprises topically applying a therapeutically effective amount of the composition of claim 1 to the eye.
